(19) United States
(12) Patent Application Publication
(10) Pub. No.: US 2010/0145211 A1
(43) Pub. Date: Jun. 10, 2010

(54) GAS FLOW SYSTEM, METER, AND METHOD

(75) Inventor: Shinji Yamamori, Tokyo (JP)

Correspondence Address: KIMBLE INTELLECTUAL PROPERTY LAW, PLLC, 1701 PENNSYLVANIA AVE., NW, SUITE 300, WASHINGTON, DC, 20006, US

(73) Assignee: Nihon Kohden Corporation, Tokyo (JP)

(21) Appl. No.: 12/546,662

(22) Filed: Aug. 24, 2009

(30) Foreign Application Priority Data
Dec. 5, 2008 (JP) 2008-311290

(51) Int. Cl. A61B 5/087; G01F 1/68

(52) U.S. Cl. 600/538; 73/204.27

(57) ABSTRACT

A system, flow meter, and method for measuring gas flow information, such as respiratory information. In an exemplary embodiment, the flow meter may include a cylindrical body that allows the respiratory gas to flow through and at least one hot wire disposed within the cylindrical body. Further, the flow meter may include a bridge circuit that includes the at least one hot wire as a resistive element and an extraction circuit that extracts a signal from the bridge circuit indicating the respiratory volume. A first filter and second filter may be disposed at an output side of the extraction circuit, and a detection circuit may be included that detects the respiratory flow rate from the output signal of the first filter and detects sound, such as snoring from the output signal of the second filter.

This application claims priority to Japanese Patent Application No. 2008-311290 filed on Dec. 5, 2008 in the Japan Patent Office, the disclosure of which is incorporated herein in its entirety by reference.

### BACKGROUND OF THE INVENTION

1. Field of the Invention

The disclosure relates generally to flow meters, and more particularly to a gas flow system, flow meter, sound detector, and method for measuring gas flow rate and sound, such as respiratory flow rate and snoring, using at least one hot-wire-type sensor.

2. Description of the Related Art

In recent years, respiratory flow rate and snoring have become basic parameters needed for diagnosing sleep apnea syndrome (hereinafter referred to as “SAS”). The widely used traditional method is to attach a sensor near the throat to detect the snoring as audio signals or as vibrating signals. This method requires a totally separate sensor aside from the sensor for respiratory flow rate, and causes the problem of creating annoying and uncomfortable feelings for the patient from attaching the sensor.

In the related art, there is also a method of utilizing a piezoelectric element that detects respiration and snoring from a living body as noted in Japanese Published Unexamined Patent Application No. 2006-212271. However, in this method, the output generated from the respiratory flow rate will vary depending on the attachment condition of the piezoelectric element, the shape of the mouth, or the size of opening of the mouth, etc. In addition, this method can only qualitatively measure the inhalation and exhalation flow rates because the piezoelectric element is exposed to the ambient air, and cannot measure quantitatively the respiratory flow rate.

For the method described above, the device to detect the snoring is used for the purpose of controlling the respiratory flow rate. In this device, the pressure sensor is used to monitor and detect the airway pressure. The detected pressure signal is filtered, and then only those frequencies contained within the specified frequency bandwidth are obtained as the filtered pressure signal. This filtered pressure signal is compared against the threshold value. The vibration of the filtered pressure signal around the threshold is detected as the first vibration, and the standard period of the first vibration is determined.

Furthermore, when there is a second vibration continuing within the filtered pressure signal and when this second vibration exceeds the threshold, the second vibration is detected and the period of the second vibration is determined. This second period is compared against the standard period in order to determine whether the second period is consistent with the standard period. If it is consistent, then the second vibration is determined as the snoring as noted in Japanese Patent Application Publication No. 2005-505329.

However, in this device, the processing is very complex as described above, and the snoring cannot be detected simply by looking at the waveform. In addition, since the respiratory signal is measured from the airway pressure, if this device is applied to a patient with a low respiratory volume, such as an infant, sufficient pressure or the vibration cannot be obtained because of weak respiration, and the snoring cannot always be captured precisely.

The present invention overcomes the aforementioned and other problems of the related art, and provides, as one aspect, a flow meter, such as a respiratory air information sensor that can appropriately detect the respiratory flow rate and snoring simultaneously.

### SUMMARY OF THE INVENTION

A first aspect of the disclosure provides a flow meter, such as a respiratory information sensor for measuring respiratory flow rate. The flow meter may comprise a body structured to allow gas to flow through; at least one hot wire installed within the body; a bridge circuit including at least one hot wire as a resistive element; an extraction circuit structured to extract one or more signals from the bridge circuit, and a detection circuit structured to detect a sound such as snoring from one or more outputs of the extraction circuit.

According to the first aspect of the invention, the flow meter may further comprise one or more filters disposed at an output side of the extraction circuit and operable to process the one or more outputs of the extraction circuit and provide the processed outputs as output signals to the detection circuit.

Further, a first of the one or more filters may be a low-pass filter and a second of the one or more filters may be one of a high-pass filter and a band-pass filter.

A second aspect of the disclosure provides a method for measuring gas flow. The method may comprise increasing a temperature of a hot wire installed within a body above ambient; flowing gas, such as respiratory gas through the body; and detecting a change in current flowing through the hot wire and corresponding gas flow rate and sound.

Further, the method may comprise determining a respiratory volume from the gas flow rate and/or detecting a direction of flow of the gas.

A third aspect of the disclosure provides a system for measuring gas flow. The system may comprise a body structured to allow gas to flow through; at least one hot wire installed within the body; a bridge circuit including the at least one hot wire as a resistive element; an extraction circuit structured to extract one or more signals from the bridge circuit, a detection circuit structured to detect a sound from one or more outputs of the extraction circuit; a processor; and a memory, wherein the processor is structured to determine an amplitude of the sound in the gas flow.

### BRIEF DESCRIPTION OF THE DRAWINGS

These and other features of this disclosure will be more readily understood from the following detailed description of the various aspects of the disclosure taken in conjunction with the accompanying drawings that depict various embodiments of the disclosure, in which:

FIG. 1 is a block diagram showing the configuration of an exemplary embodiment of a flow meter, such as a respiratory information sensor according to the present invention;

FIG. 2 is a diagram showing an exemplary embodiment of a bridge circuit used in a flow meter according to the present invention;

FIG. 3 is a diagram showing another exemplary embodiment of a bridge circuit used for differentiating the gas flow direction according to the present invention; and

FIG. 4 is a diagram showing waveforms of a respiratory volume and snoring that are displayed by an exemplary embodiment of the flow meter according to the present invention.

It is noted that the drawings of the disclosure are not to scale. The drawings are intended to depict only typical aspects of the disclosure, and therefore should not be considered as limiting the scope of the disclosure. In the drawings, like numbering represents like elements between the drawings.

### DETAILED DESCRIPTION OF THE INVENTION

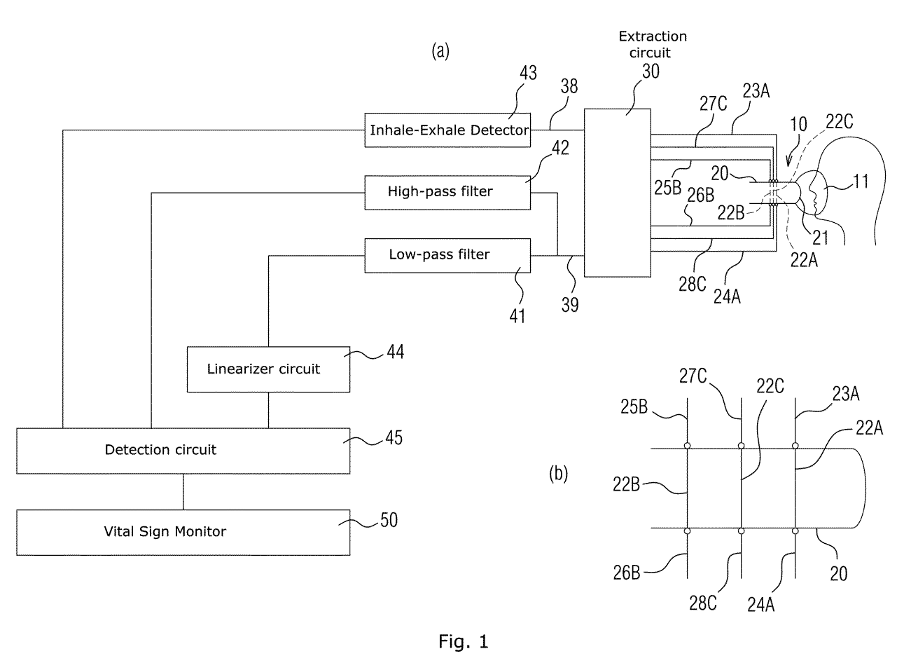

The present invention will now be described more fully with reference to the accompanying drawings. A flow meter, such as a respiratory information sensor according to an exemplary embodiment of the invention is shown in FIG. 1. This embodiment of the respiratory information sensor utilizes the sensor section 10 that is configured such that the opening 21 at one end of the cylindrical body 20 is connected to the mask 11. The mask 11 covers the mouth and nose of the patient and is configured to have all of the inhaled and exhaled gas flowing through the cylindrical body 20.

FIG. 1(a) shows an overall configuration of the respiratory information sensor and FIG. 1(b) shows an enlarged diagram of the inside of the cylindrical body 20. In the inside of the cylindrical body 20 where the respiratory flows through, three hot wires 22A, 22B and 22C are placed and aligned adjacently in the longitudinal direction of the cylindrical body 20. Of course, longitudinal placement of the hot wires does not preclude other configurations, such as placing the hot wires in a transverse direction, and the body may also take on some other shape than cylindrical. The hot wires 22A, 22B and 22C may comprise, for example, platinum and tungsten. The elements of these materials generate heat and change their resistance values when electricity is applied. In the embodiment, the hot wire 22A is positioned closest to the mask 11, the hot wire 22B is positioned farthest away from the mask 11, and the hot wire 22C is positioned between the hot wires 22A and 22B, although other placements of the wires or use of less or more hot wires is not prohibited. The lead wires 23A and 24A attached to the ends of the hot wire 22A, the lead wires 25B and 26B attached to ends of the hot wire 22B, and the lead wires 27C and 28C attached to the ends of the hot wire 22C are all connected to the extraction circuit 30.

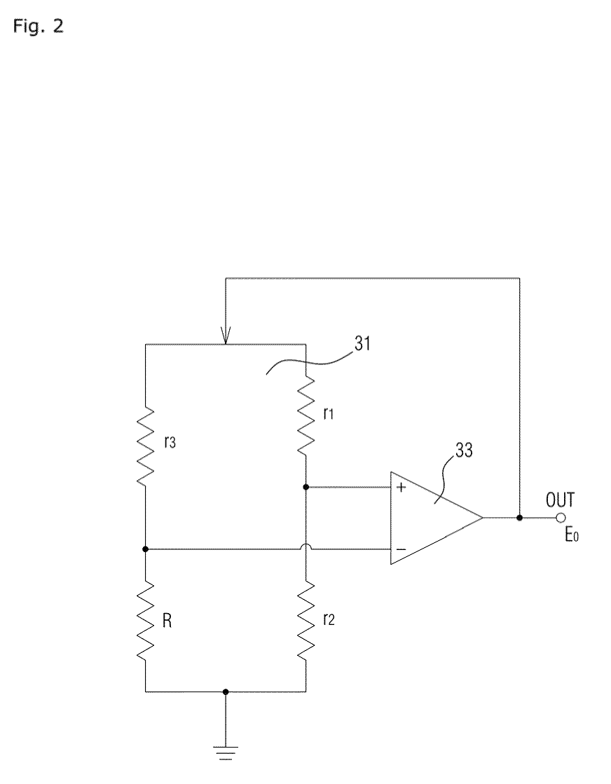

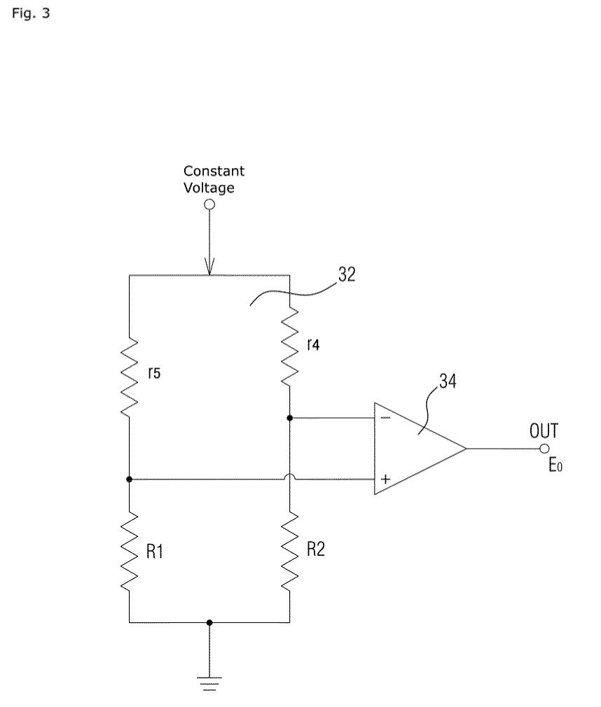

The extraction circuit 30 may contain the bridge circuit 31 as shown in FIG. 2, and the bridge circuit 31 may have the hot wire 22C as one of the resistive elements. From this bridge circuit 31, the signal indicating the flow rate of the inhaled or exhaled gas is obtained as the resistance of the hot wire 22C varies responding to the inhaled or exhaled gas flowing upon it. The extraction circuit 30 of this embodiment additionally may have the bridge circuit 32 as shown in FIG. 3 to detect the direction of the flow differentiating between inhalation or exhalation.

The bridge circuit 31 shown in FIG. 2 may be known as a hot wire constant temperature circuit. The resistor R shown in the bridge circuit 31 represents the resistance of the hot wire 22C, and other resistors r1 through r3 are fixed value resistors. The feedback current from the output of the operational amplifier 33 is fed to the junction of the resistors r1 and r3. The junction of the resistors R and r2 is connected to the ground, and thus, the current flows through the bridge. The resistive values of the fixed resistors are set so that the bridge 31 attains the equilibrium state when the resistance of R is heated to a certain temperature, 400 degrees C. for example, under a current application.

The junction of the resistors r1 and r2 may be connected to the non-inverting input terminal of the operational amplifier 33. The junction of the resistors r3 and R may be connected to the inverting input terminal of the operational amplifier 33. When the bridge circuit 31 becomes unbalanced, the difference at the inputs may be amplified to obtain the output signal Eo. The current from the output signal Eo may be fed back to the bridge circuit 31, and the current may continue to increase until the bridge 31 attains the equilibrium state. In the pre-measurement state of the cylindrical body 20, the output of the operational amplifier 33 may continue to increase and the current to the bridge circuit 31 may also continue to increase until the resistor R is heated and increases its resistance value to the set point, 400 degrees C. for example, and the bridge 31 attains the equilibrium state.

Once the extraction circuit 30 attains the measurement state, and if the respiration flows within the cylindrical body 20, the resistor R of the hot wire 22C may be cooled and the resistance value of the resistor R will change and the bridge circuit 31 will be unbalanced. The differential voltage created by the unbalanced bridge circuit 31 will be amplified to produce the greater output voltage that is in turn fed back to increase the current to the bridge circuit 31 until the bridge circuit 31 attains the equilibrium state again. Since the resistor R may be cooled in proportion to the respiratory flow rate, the output signal Eo of the operational amplifier 33 can be converted to the respiratory flow rate by computation considering the inner diameter of the cylindrical body 20.

The bridge circuit 32 shown in FIG. 3 is for differentiating the flow direction. The hot wire 22A (R1 in FIG. 3) and the hot wire 22B (R2 in FIG. 3) used in the bridge circuit 32 may be the same type of hot wire as the hot wire 22C. However, the applied voltage to the bridge may be set so that the temperature of the hot wires 22A and 22B hardly increases. The hot wires 22A and 22B may be used to perform the function of thermo-sensitive hot wires.

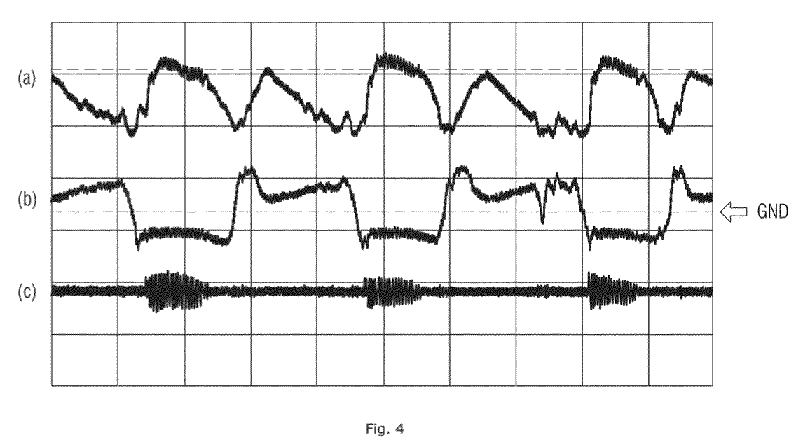

As described earlier, inside the cylindrical body 20, three hot wires 22A, 22B and 22C may be placed and aligned adjacently in the longitudinal direction of the cylindrical body 20. The temperature of the hot wire 22C is maintained high and the inhaled or exhaled gas flowing upon it will be warmed up, and the hot wire (either 22A or 22B) situated at the downstream side of the hot wire 22C will receive warm air. As a result, the resistance of the hot wire (either 22A or 22B) will increase, causing the bridge circuit 32 to become unbalanced, and the unbalanced voltage will be output. The output unbalanced voltage will move in the plus or minus direction as shown in FIG. 4B depending on the direction of the flow, and thus the direction can be identified. In other words, it is possible to detect whether inhaled or exhaled gas is flowing within the cylindrical body 20. The output of the operational amplifier 34, to which the bridge circuit 32 having the hot-wires 22A and 22B is connected, may be routed to the inhalation/exhalation differentiation circuit 43, such as an inhale-exhale detector, via the output line 38. Based on the detection result from the inhalation/exhalation differentiation circuit 43, processing, such as filtering and zero-adjustment, is performed, and then the output may be routed to the detection circuit 45.

The output of the operational amplifier 33, to which the bridge circuit 31 having the hot wire 22C is connected, may be output via the output line 39 that branches out in two directions. One branch of the output line 39 may be routed to the low-pass filter 41 with a cutoff frequency of approximately 20 Hz and to the linearizer circuit 44 for the purpose of performing the linear approximation. The other branch of the output line 39 may be routed to the high-pass 42 with a cutoff frequency of approximately 20 Hz. Note that the high-pass filter 42 may be replaced by for example, a band-pass filter. It is noted that a frequency over 150 Hz may include sounds other than snoring, in which situation a band pass filter from 20 Hz to 150 Hz may be used in an exemplary embodiment.

FIG. 4(a) shows an exemplary embodiment of the waveform of the respiratory flow rate after processing through the low-pass filter 41, and FIG. 4(b) shows an exemplary embodiment of the waveform related to the direction of the flow after processing by the inhalation/exhalation differentiation circuit 43. FIG. 4(c) shows an exemplary embodiment of the waveform related to the snoring after processing through the high-pass filter 42.

As evidenced by FIG. 4(c), the signal that has passed through the high-pass filter 42 may have a waveform having higher vibration amplitude within a shorter duration over a period. This enables the observer to capture the occurrence of the snoring visually.

The output signal from the low-pass filter 41 may be first linearly approximated by the linearizer circuit 44, then routed to the detection circuit 45 where the signal may be identified as either in the inhalation phase or the exhalation phase based on the other signal from the inhalation/exhalation differentiation circuit 43 and converted to the respiration flow rate signal. In addition, the detection circuit 45 may integrate the respiration flow rate signal and compute the respiration volume signal. Then the respiration flow rate signal, the respiration volume signal, and the snoring signal may be sent to the vital information measurement device 50, such as a vital sign monitor. The vital information measurement device 50 may be configured with instruments such as a computer that would receive the signals from the detection circuit 45 and perform the signal processing for generating waveform display images, etc.

The vital information measurement device 50 may receive the signals from the detection circuit 45, create the waveform images, and display them.

In the earlier descriptions, a constant temperature hot wire bridge is utilized for the bridge circuit 31. However, a constant current type of bridge for the bridge circuit 32 may be used, in which the resistor R1 is configured by the hot wire 22C, to detect the respiratory flow rate and sound, such as snoring.

It is also acceptable in the extraction circuit 30 to configure the bridge circuit 31 and the bridge circuit 32 by using two bridge circuits 31.

When two bridge circuits 31 are used, the output voltage from the downstream bridge circuit 31 will be smaller than the output voltage from the upstream bridge circuit 31 even though the same flow rate is applied to both bridges. This is because less heat, thus less temperature, is taken away from the downstream resistor being influenced by the heat from the upstream hot wire. This difference in the output voltages may be used to detect the direction of the flow. For detecting the flow rate, the upstream bridge circuit is used. The respiratory information sensor will be configured such that the output signal Eo from the upstream bridge circuit 31 is fed through one of the low-pass filters to obtain the respiratory flow rate waveform images, and is fed through one of the high-pass filters to obtain the snoring waveform images. Other exemplary embodiments may be configured similarly to the configuration already described earlier.

Note that the processing of information in the present invention may be performed by a processor that may include a computer-readable medium as known to those of ordinary skill in the art.

While this invention has been particularly shown and described with reference to exemplary embodiments thereof, it will be understood by those skilled in the art that various changes in form and details may be made therein without departing from the spirit and scope of the invention as defined by the appended claims.

What is claimed is:

1. A flow meter, comprising:
a body structured to allow gas to flow through;
at least one hot wire installed within the body;
a bridge circuit including the at least one hot wire as a resistive element;
an extraction circuit structured to extract one or more signals from the bridge circuit, and
a detection circuit structured to detect a sound from one or more outputs of the extraction circuit.

2. The flow meter according to claim 1, further comprising one or more filters disposed at an output side of the extraction circuit and operable to process the one or more outputs of the extraction circuit and provide the processed outputs as output signals to the detection circuit.

3. The flow meter according to claim 2, wherein a first of the one or more filters is a low-pass filter and a second of the one or more filters is one of a high-pass filter and a band pass filter.

4. The flow meter according to claim 2, wherein the one or more filters comprise a digital filter, an electric circuit, or a combination thereof.

5. The flow meter according to claim 2, wherein the one or more filters include a first filter and a second filter, the first filter outputting a first signal of the one or more outputs used by the detection circuit to detect a respiratory flow rate, and the second filter outputting a second signal of the one or more outputs used by the detection circuit to detect the sound.

6. The flow meter according to claim 1, further comprising a second bridge circuit, wherein output voltage from the bridge circuit disposed downstream to the other one is smaller.

7. The flow meter according to claim 1, wherein variations in resistive values of the at least one hot wire are due to at least one of a change in temperature and a change in an amount of current provided to the bridge circuit.

8. The flow meter according to claim 1, wherein a signal of the one or more signals is used to determine a respiratory volume, which is reflected by changes in a resistive value of the at least one hot wire as the gas flows through.

9. The flow meter according to claim 1, wherein the body has a cylindrical shape.

10. The flow meter according to claim 1, further comprising an inhalation/exhalation differentiation circuit structured to detect a direction of flow of the respiratory gas, the inhalation/exhalation differentiation circuit including a second hot wire disposed side-by-side in a longitudinal direction of the body.

11. The flow meter according to claim 1, further comprising an inhalation/exhalation detection circuit structured to detect a direction of flow of the respiratory gas, the inhalation/exhalation detection circuit including a second at least one hot wire disposed in at least one of a front and back position with respect to the other at least one hot wire, and in a longitudinal direction of the cylindrical body.

12. The flow meter according to claim 1, further comprising a mask structured to cover at least one of a mouth and a nose of a living body, wherein the mask is connected to an opening at one end of the cylindrical body.

13. The flow meter according to claim 1, wherein the detection circuit is operable to determine a respiratory volume from a respiratory flow rate.

14. A method for gas flow, the method comprising:
increasing a temperature of a hot wire installed within a body above ambient;
flowing gas through the body; and
detecting a change in current flowing through the hot wire and corresponding gas flow rate and sound.

15. The method according to claim 14, further comprising determining a gas volume from the gas flow rate.

16. The method according to claim 14, further comprising detecting a direction of flow of the gas.

17. The method according to claim 14, further comprising determining a gas volume, which is reflected by changes in a resistive value of the hot wire as the gas flows through the body.

18. The method according to claim 14, further comprising performing one of high-pass and band-pass filtering by a digital filter, an electric circuit, or a combination thereof.

19. The method according to claim 14, further comprising displaying results of the detection of the sound in a form of a sound waveform.

20. A system for measuring gas flow information, the system comprising:
a body structured to allow gas to flow through;
at least one hot wire installed within the body;
a bridge circuit including the at least one hot wire as a resistive element;
an extraction circuit structured to extract one or more signals from the bridge circuit;
a detection circuit structured to detect a sound from one or more outputs of the extraction circuit;
a processor; and
a memory, wherein the processor is structured to determine an amplitude of the sound in the gas flow.
